(19) United States
(12) Patent Application Publication
(10) Pub. No.: US 2026/0053845 A1
(43) Pub. Date: Feb. 26, 2026

(54) LIPOSOMAL FORMULATION AND USE IN A COMBINATION PRODUCT AS AN ANTI-TUMOUR THERAPY

(71) Applicants: Institut National de la Santé et de la Recherche Médicale (INSERM), Paris (FR); Centre National de la Recherche Scientifique (CNRS), Paris (FR); Université Claude Bernard Lyon 1, Villeurbanne (FR); Hospices Civils de Lyon, Lyon (FR); Centre Léon Bérard, Lyon (FR)

(72) Inventors: Charles DUMONTET, Venissieux (FR); Abdelkamel CHETTAB, Lyon (FR)

(73) Assignees: Institut National de la Santé et de la Recherche Médicale (INSERM), Paris (FR); Centre National de la Recherche Scientifique (CNRS), Paris (FR); Université Claude Bernard Lyon 1, Villeurbanne (FR); Hospices Civils de Lyon, Lyon (FR); Centre Léon Bérard, Lyon (FR)

(21) Appl. No.: 19/342,962

(22) Filed: Sep. 29, 2025

Related U.S. Application Data
(63) Continuation-in-part of application No. 16/760,215, now Pat. No. 12,433,838.

(30) Foreign Application Priority Data
Oct. 30, 2017 (FR) 1760209

(51) Int. Cl. A61K 31/739; A61K 9/127; A61K 31/475; A61K 31/7068; A61K 38/14; A61K 39/395; A61P 35/00

(52) U.S. Cl. CPC A61K 31/739; A61K 9/127; A61K 31/475; A61K 31/7068; A61K 38/14; A61K 39/39558; A61P 35/00

(57) ABSTRACT

A pharmaceutical combination product including:
a liposomal formulation consisting of one or more liposomes each encapsulating a bacterial lipopolysaccharide (LPS) as a single active ingredient; and
at least one anti-tumor compound chosen from the group consisting of: a therapeutic antibody, a chemotherapy agent, and an immunotherapy agent.

Methods for treating a tumor comprising administering to a patient having said tumor an effective amount of said pharmaceutical combination product.

### CROSS-REFERENCE TO RELATED APPLICATIONS

This application is a Continuation-in-Part of U.S. patent application Ser. No. 16/760,215, filed on Apr. 29, 2020, now U.S. Pat. No. 12,433,838, issued on Oct. 7, 2025, which is a U.S. National Stage Application pursuant to 35 U.S.C. § 371 of International Patent Application PCT/FR2018/052695, filed on Oct. 30, 2018, and published as WO 2019/086806 on May 9, 2019, which claims priority to French Patent Application 1760209, filed on Oct. 30, 2017, all of which are incorporated herein by reference in their entireties for all purposes.

### FIELD OF THE INVENTION

The present invention relates to the pharmaceutical field, and more particularly to the oncology field, in particular to the cancer treatment field. More particularly, the present invention relates to a pharmaceutical composition for treating a tumour, this composition being used as such or as an adjuvant composition of another therapeutic compound. The present invention is also related to a pharmaceutical combination product.

### STATE OF THE ART

Cancer is a disease characterised by the uncontrolled multiplication of cells within an organism, this multiplication being related to genetic mutations affecting the DNA of its cells. These mutations appear in a spontaneous or induced manner, as the result of an exposure to mutagenic agents, or even have been transmitted in an hereditary way.

Cancer cells of various origins, having an excessive cellular proliferation, give rise to a “tumour”, that is a tissue mass tending to persist and grow within the original tissue, and possibly to disseminate in other tissues.

In the case of circulating, in particular blood, cancer cells, these cells are characterised by an anarchical, uncontrolled growth and division capability.

Today, many anti-tumour therapies exist. Without limitation, the available treatments comprise surgical tumour ablation, chemotherapy (administration of cytotoxic drugs for destroying cancer cells), radiation therapy (tumour irradiation), hormone treatments, anti-angiogenic treatments and immunotherapy.

Immunotherapy is a treatment which consists in administrating biological substances, usually produced by the immune system, in order to boost and/or stimulate immune defences of an organism. Indeed, it has been observed that, at the onset of a tumour, the affected organism generates on its own a immune response to this tumour, the tumour cells being recognised as such by the immune system. This immune response is however generally insufficient to get rid of said tumour. The purpose of the immunotherapy is thus to sustain and/or replace this insufficient biological response.

Biological substances used in immunotherapy are for example antibodies, in particular monoclonal antibodies, cytokines, interleukins, interferons and generally any immunostimulant compound.

For each type of tumour, clinicians decide on the treatment(s) to be applied depending on previously validated medical protocols, and each patient's specificities. Currently available treatments can be further improved insofar as none has a full efficiency, nor is adapted to any patient type.

In immunotherapy, studies have been made in order to potentiate beneficial effects of a therapeutic antibody administration. For example, patent application WO 2013/129936 describes the therapeutic use of a combination consisting of an antibody and an immuno-modulator encapsulated in a particulate or vesicular material. The encapsulated immuno-modulator is in particular a cytokine wrapped into a liposome, the co-administration of which stimulates beneficial effects of the antibody.

### Liposomes

A liposome is an artificial vesicle formed by concentric lipid bilayers, trapping molecules between them. Liposomes are more usually comprised of phospholipids, of a single or several types. These phospholipids are organised in a thermodynamically stable state such that the polar heads are gathered and enable a bilayer to be established. Liposomes are nano-size structures.

Liposomes can retain several types of compounds regardless of whether they are water-soluble (encapsulated in the aqueous phase) or oil-soluble or amphiphilic (packed in the lipid bilayer).

The use of liposomes in the pharmaceutical industry, as a vector for various molecules of biological interest, has been the object of many works and several drugs of this type are currently approved for an intravenous administration.

Indeed, encapsulating active substances into liposomes enables said substances to be protected. It also enables the toxic action of the substances to be limited, and their release rate to be regulated. In addition, liposomes allow passage of water-soluble substances through the hydrophobic membrane of the cells.

Liposomes are increasingly used in therapy as drug vectors. Their primary use is targeting active ingredients.

### Bacterial Lipopolysaccharide (LPS)

The bacterial lipopolysaccharide (LPS) is the main component of the external membrane of Gram-negative type bacteria. LPS has high immunostimulatory capabilities: its presence in an organism generates the stimulation of the entire immune system, in particular via the secretion of pro-inflammatory cytokines, in order to respond to the bacterial contamination. Because of this strong immune reaction, injecting LPS in its soluble form can be toxic, or even lethal at a high dose, for a mammalian organism. LPS induces in humans reactions such as hyperthermia, red blood cell aggregation, and septic shock.

The LPS molecule comprises three entities: the lipid A, the nucleus and the antigen O. The lipid A which represents the most toxic LPS part is highly conserved, the nucleus is of very low variability whereas the antigen O is specific to the bacterial species from which comes the LPS.

The action mode and signal pathways of LPS are well known. Its target cells are mainly macrophages, monocytes, granulocytes and epithelial cells. Once released in the organism, the LPS binds to the LBP (LPS Binding Protein) protein synthesised by the liver, which enables its presentation and binding to the membrane receptor CD14 of the monocytes. The LPS is thereby also associated with a membrane co-receptor MD2 and with the receptor TLR 4 in its homodimerised form. The formation of this membrane complex induces activation of the MAP-Kinase pathway and secretion of pro-inflammatory cytokines in the cell.

In basic research, LPS is used in vitro and in vivo, to induce an inflammation related to a strong secretion of pro-inflammatory cytokines by monocyte or macrophage type cells.

In therapy and mainly in prophylaxy, LPS or its entity ‘lipid A’ is used as a vaccinal adjuvant, to stimulate an immune response targeted against an antigen co-administrated with said LPS.

### Liposomal Formulation of LPS

It has been shown that encapsulating LPS into liposomes minimises these pro-inflammatory effects both in vitro and in vivo (Bakouche et al., 1987; Dijkstra et al., 1989).

In anti-tumour therapy, LPS has been used as an adjuvant, to stimulate the immune response to a specific antigen: Neidhart et al. (Vaccine, 2004) have described a treatment method for patients with colorectal cancer, comprising administrating a therapeutic vaccine consisting of a liposomal formulation comprising both a recombinant KSA protein and monophosphoryl lipid A (active component of LPS); most patients treated with this therapeutic vaccine have developed a specific immunity against the KSA protein.

A LPS liposomal formulation has thus already been used within the scope of the preparation of anti-tumour vaccine compositions, the LPS being used for its adjuvant properties. Its co-formulation with an antigen enables the specific immune response against this antigen to be optimised.

However, to date, it has never been contemplated to use LPS as a single active ingredient, in a liposomal formulation, for use in therapy.

Additionally, the association of a liposomal formulation containing exclusively LPS with at least one cytotoxic compound has never been contemplated, as well as the use of said association to treat tumours.

### DISCLOSURE OF THE INVENTION

Encapsulating LPS into liposomes enables its toxic effects to be minimised and thus this liposomal formulation to be used in therapy, in particular within the scope of an anti-tumour therapeutic treatment by systemic way.

The present invention relates to a pharmaceutical combination product comprising:
a liposomal formulation exclusively containing a LPS; and
at least one cytotoxic compound, as for example a therapeutic antibody or a chemotherapy compound.

The invention also relates to said therapeutic combination product for simultaneous, separate or sequential use as an anti-tumour therapy.

The present invention also relates to a liposomal formulation exclusively containing a bacterial lipopolysaccharide (LPS) for use as an anti-tumour therapy.

More generally, a liposomal formulation exclusively containing a bacterial lipopolysaccharide (LPS) for use as a drug is described.

Specifically, the present invention relates to a pharmaceutical combination product comprising:
a liposomal formulation consisting of one or more liposomes each encapsulating a bacterial lipopolysaccharide (LPS) as a single active ingredient; and
at least one anti-tumor compound chosen from the group consisting of: a therapeutic antibody, a chemotherapy agent, and an immunotherapy agent.

### DESCRIPTION OF THE FIGURES

FIG. 1. Monitoring of tumour volumes of lymphoma RL xenografted mice (CD20+ human B lymphoma cells reference ATCC CRL-2261) not treated (control) or treated with non-encapsulated LPS, rituximab, empty liposomes, a LPS liposomal formulation and a combination of rituximab+LPS liposomal formulation.

FIG. 2. Weights of treated mice spleens: control mice (CTRL), treated with empty liposomes (Empty-Lipo), treated with the antibody Rituximab (Ritux), treated with non-encapsulated LPS (LPS), treated with a LPS liposomal formulation (Lipo-LPS), and treated with a combination of antibodies Rituximab and a LPS liposomal formulation (Ritux+Lipo-LPS).

FIG. 3. Amount of NK cells in treated mice spleens: control mice (control), treated with the antibody Rituximab (Ritux), treated with non-encapsulated LPS (LPS), treated with empty liposomes (Empty-Lipo), treated with a LPS liposomal formulation (Lipo-LPS), and treated with a combination of antibodies Rituximab and a LPS liposomal formulation (Ritux+Lipo-LPS).

FIG. 4. Monitoring of tumour volumes of RL lymphoma xenografted mice treated with (i) Rituximab, (ii) a combination of Rituximab with a LPS liposomal formulation, or (iii) a combination of Rituximab with non-encapsulated LPS.

FIG. 5. Monitoring of tumour volumes of MDA-MB-231 cancer cells xenografted scid mice, treated with the LPS liposomal formulation (Lipo LPS, grey curve) or not treated (CONTROL).

FIG. 6. Rat osteosarcoma cells cultured in vitro, incubated with:
macrophages stimulated for 16 h with 100 ng/mL Lipo-LPS WO (LPS-Biosciences), or
5 μM etoposide, or
a combination of both.

The control cells have been exposed to the vehicle only, containing neither macrophages nor etoposide.

The apoptosis rate of the cancer cells has been monitored by a labelling with the IncuCyte® Caspase-3/7 compound.

FIGS. 7A-7B. Monitoring of tumor volumes (in mm³) over time of murine B-cell lymphoma cells (A20) grafted in BALB/c mice, further treated with (i) vehicle (control), (ii) liposomal formulation HEPHA-440 (25 μg i.v.), (iii) monoclonal antibodies against mouse CD20 (anti-CD20 mAb—clone 18B12-12.5 mg/kg i.p.); and (iv) a combination of HEPHA-440 with monoclonal antibodies anti-CD20. FIG. 7A. Mean tumor volume for each of the four conditions are represented in the same graph. FIG. 7B. For each condition, tumor volumes of 8 tested mice are represented; the percentage of mice in complete remission (CR) is indicated.

FIGS. 8A-8C. Monitoring of tumor volumes (in mm³) over time of human RL lymphoma xenografted in SCID mice, treated with (i) vehicle (control), (ii) monoclonal antibodies against human CD20 (GA101—30 mg/kg i.p.) and (iii) a combination of HEPHA-440 with monoclonal antibodies anti-CD20. FIG. 8A. Mean tumor volume for each of the three conditions are represented in the same graph. FIG. 8B. For each condition, tumor volumes of 6 tested mice are represented. FIG. 8C. Survival rates for each condition.

FIGS. 9A-9C. Monitoring of tumor volumes (in mm³) over time of mouse colon carcinoma cells (MC38) grafted in C57BL/6 mice treated with (i) vehicle (control—Black line), (ii) antibodies against mouse PD-I (aPD-1, RPM1-14-12.5 mg/kg in i.p.—Black dotted lines), (iii) liposomal formulation HEPHA-440 (10 μg i.v.) (Light grey line), and (iv) a combination of HEPHA-440 with antibodies aPD-1 (Light grey dotted line). FIG. 9A. Mean tumor volume for each of the four conditions are represented in the same graph. FIG. 9B. For each condition, tumor volumes of 6 tested mice are represented; the number of mice in complete remission (CR) or partial remission (PR) is indicated. FIG. 9C. Survival rates for each condition.

FIGS. 10A-10D. Monitoring of tumor cells viability incubated in vitro in presence of effector cells: (i) in control conditions (black), (ii) with HEPHA-440 (light grey), (iii) with a chemotherapy agent (Black stripes), and (iv) with a combination of a chemotherapy agent and HEPHA-440 (light grey stripes). (10A) Mean size of human Saos-2 spheroids in each of the four conditions with human THP-1 macrophages as effector cells and doxorubicin as a chemotherapy agent. FIGS. 10B, 10C, 10D. Mean percentage of viable MC38 colorectal tumor cells in each the four conditions with mouse splenocytes as effector cells and chemotherapy agents: gemcitabin (FIG. 10B), irinotecan (FIG. 10C) and bleomycin (FIG. 10D).

### DETAILED DESCRIPTION OF THE INVENTION

The present invention is based on the identification of new immuno-stimulatory properties for the bacterial lipopolysaccharide (LPS). Because of its toxicity, this immunostimulant compound cannot be used as such. LPS has already been used as an adjuvant in vaccines, under liposomal formulation containing an antigen and LPS.

The inventors have highlighted the fact that a LPS liposomal formulation, containing no other compounds and in particular no antigen, can be used as a drug, and more particularly that this formulation has therapeutic beneficial effects within the scope of an anti-tumour treatment.

According to a first aspect, the invention relates to a liposomal formulation exclusively containing a bacterial lipopolysaccharide (LPS) for use as a drug.

According to a second aspect, the invention relates to a liposomal formulation exclusively containing a bacterial lipopolysaccharide (LPS) for use as an anti-tumour therapy.

In other words, the present invention relates to a pharmaceutical formulation consisting in a bacterial LPS encapsulated into liposomes, for use in therapy, in particular for use as an anti-tumour therapy.

According to a third aspect, the invention relates to a pharmaceutical combination product comprising:
a liposomal formulation exclusively containing a LPS, and
at least one cytotoxic compound, notably an anti-tumor compound.

More specifically, the present invention concerns a pharmaceutical combination product comprising:
a liposomal formulation consisting of one or more liposomes each encapsulating a bacterial lipopolysaccharide (LPS) as a single active ingredient; and
at least one anti-tumor compound chosen from the group consisting of: a therapeutic antibody, a chemotherapy agent, and an immunotherapy agent.

According to a fourth aspect, the invention relates to said therapeutic combination product, for use as an anti-tumour therapy.

### Definitions

For the purposes of the invention, by “cancer”, it is meant a pathology characterised by the presence in an organism of cancer cells, formed by transformation of initially normal cells of the organism afflicted by this pathology. A living organism having such cancer cells is diagnosed as being afflicted with cancer. There are approximately 200 different types of cancers, depending on the tissue where the first tumour called a primary tumour is developed.

For the purposes of the invention, by “tumour” it is meant a tissue mass from an excess cell proliferation of cancer cells, this tissue mass tending to persist and grow in a non-regulated and autonomous way towards the organism.

The present invention relates to all types of tumours, but more particularly malignant tumours. Malignant tumours have usually a rapid growth, and have a tendency to relapse after local eradication. Malignant tumours are poorly limited, non-encapsulated, and their perimeters are uneven.

The present invention relates to the treatment of primary tumours and secondary tumours, coming from the metastatic dissemination of a primary tumour.

Tumours are generally classified depending on their original tissue: there are for example skin, bone, or blood cell tumours.

Two main categories of tumours have been defined:
the so-called “solid” tumours are developed in tissues such as skin, mucosae, bones and organs. These are the most frequent tumours: they account for 90% of human cancers.

Among solid tumours, there are carcinomas, coming from epithelial cells (skin, mucosae, glands); and sarcomas, coming from connective tissue cells.
The so-called “liquid” tumours come from blood cells and are not property tissue excrescences, but are characterised by the presence of cancer blood cells having an anarchical, uncontrolled growth and division capability.

Among liquid tumours, there are leukemias (blood and bone marrow cancers), characterised by the anarchical multiplication of white blood cell precursor cells in the bone marrow; and lymphomas (lymphatic system cancers) which affect lymphocytes.

According to a particular aspect of the invention, the tumour treated is a liquid tumour or a solid tumour.

According to another particular aspect of the invention, the tumour treated is chosen from the group consisting of: a breast tumour, a lung tumour, a skin tumour (melanoma), a blood tumour (leukemia), a bone tumour and a lymphoma.

### Cellular Immunotherapy

By “anti-tumour treatment” or “anti-tumour therapy” or “anti-tumour therapy use”, it is meant for the purposes of the invention a therapeutic treatment for reducing the volume, inhibiting growth, decreasing agressivity, modifying malignant functional characteristics, and/or get rid of a tumour present in an organism.

To determine and monitor the efficiency of an anti-tumour treatment, an indicative parameter is the progression of the tumour size or volume within the organism, over time. In laboratory animals, the tumour size is most often measured after sacrifying the animals. In patients, the tumour size could be measured in vivo by non-invasive imaging techniques, well known to those skilled in the art.

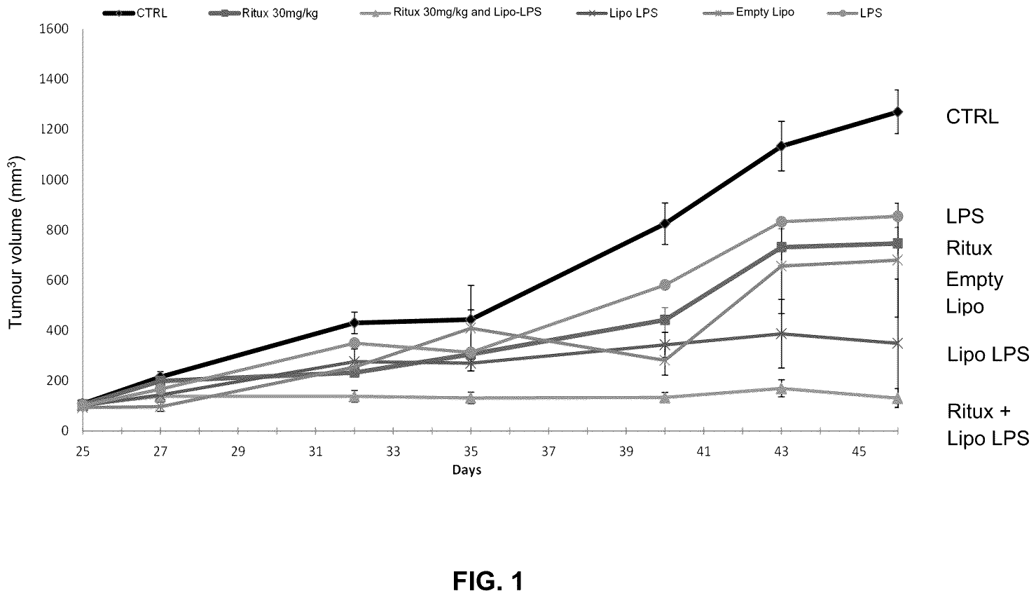

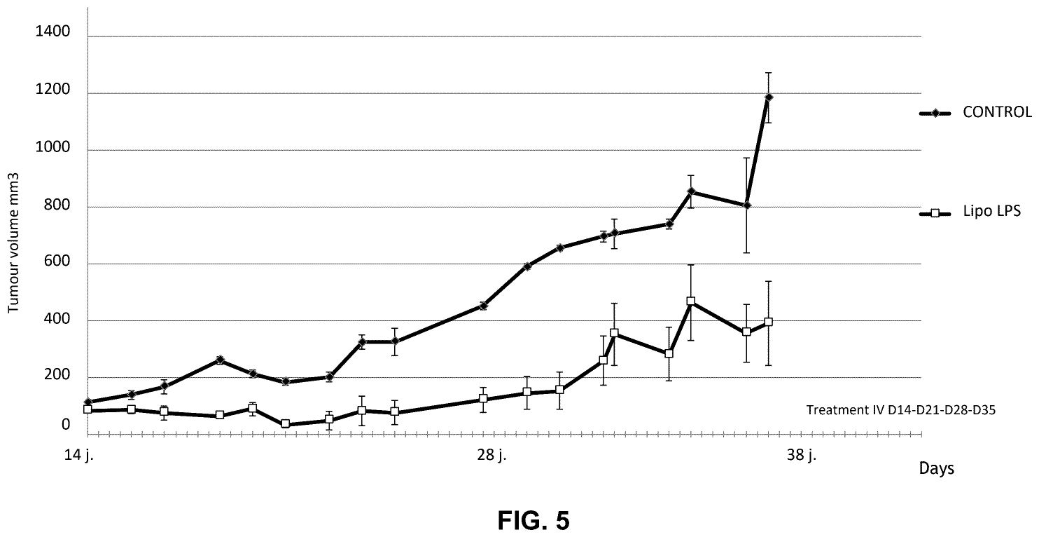

As is illustrated in FIGS. 1 and 5, the liposomal formulation exclusively containing a LPS enables the development of a human tumour xenografted in mice to be significantly reduced, in comparison with the tumour development observed in control, untreated mice.

FIG. 1 shows the results obtained with mice xenografted with RL lymphoma cells, and FIG. 5 shows the results obtained with mice having breast cancer tumours (formed from MDA-MB361 cells).

The present invention relates in particular to a liposomal formulation exclusively containing a LPS for use as an immunotherapy agent.

The present invention also relates to a liposomal formulation exclusively containing a LPS for use as an agent stimulating the innate immune system.

More particularly, the present invention relates to a liposomal formulation exclusively containing a LPS for use as an anti-tumour cell immunotherapy agent.

Immunotherapy is a therapeutic approach consisting in stimulating internal immune functions of an organism afflicted with a cancer, for the immune system of the organism to be capable of inhibiting growth or even get rid of a tumour developing therewithin.

Among immune operators involved in the recognition and destruction of cancer cells, the ‘Natural Killer’ cells designated NK cells hereinafter can be mentioned. These are lymphocytes capable of recognising a tumour tissue, infiltrating it and exerting a specific cytotoxicity to tumour cells.

The anti-tumour cellular immunotherapy is a therapeutic approach consisting in stimulating NK cells, their development and/or activity, such that they recognise and lyse tumour cells.

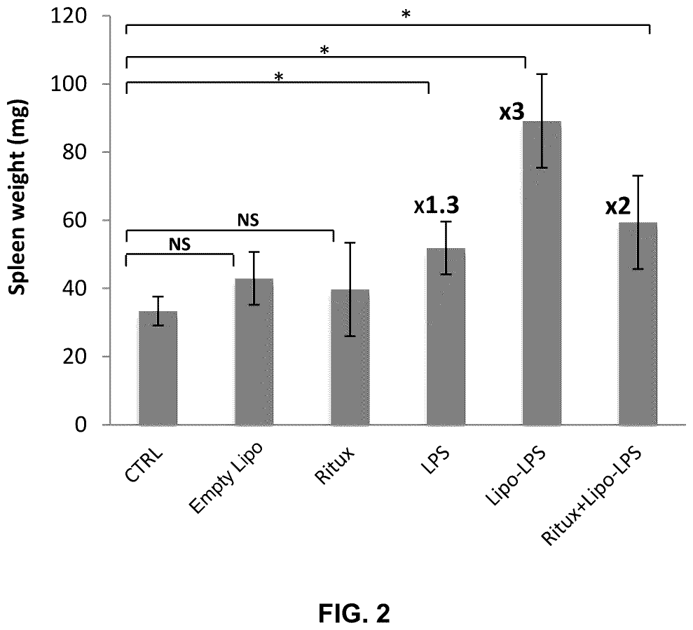

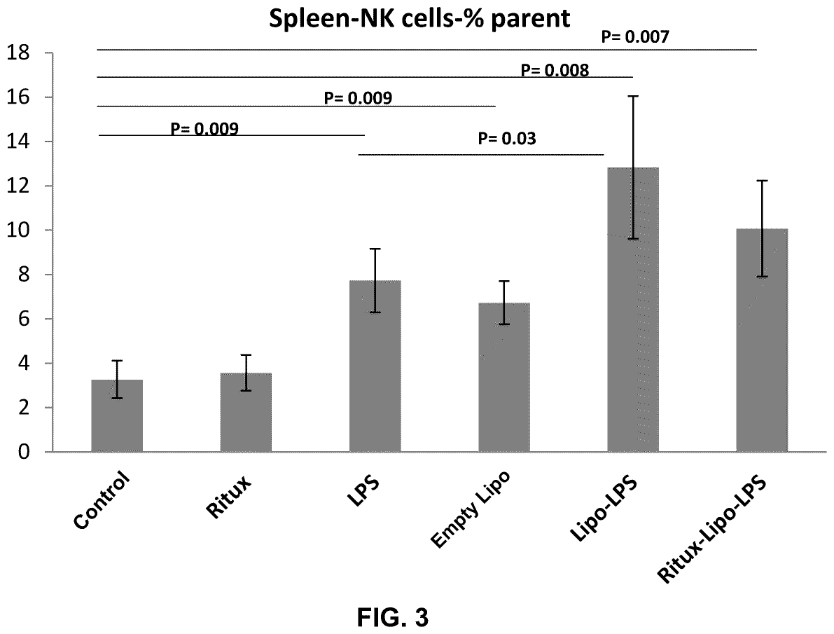

As is illustrated in FIGS. 2 and 3, the administration to mice of a liposomal formulation exclusively containing a LPS significantly increases the number of NK cells present in the animal spleen.

Thus, it appears that a liposomal formulation exclusively containing a LPS stimulates multiplication of NK cells, and thus acts as a cellular immunotherapy agent, by promoting the cytotoxic cellular response to the tumour cells.

### Liposomal Formulation

For the purposes of the invention, a liposomal formulation designates a composition comprising liposomes encapsulating an active ingredient, said active ingredient being designated as being “encapsulated” or even “contained in a liposomal formulation”.

In the present application, the terms “LPS liposomal formulation”, “liposomal formulation exclusively containing a LPS”, “liposomes-LPS” and “LPS encapsulated in liposomes” are used indifferently and all of them designate the same formulation as defined above, namely a formulation/composition consisting of liposomes encapsulating a bacterial lipopolysaccharide as a single active ingredient.

The liposomes, also designated as liposomal particles, are vesicles in which a lipid phase consisting of a bilayer of amphiphilic molecules, such as phospholipids or cholesterol, traps an aqueous inner phase.

Unilamellar liposomes, which comprise a single lipid bilayer are differentiated from multilamellar liposomes which comprise several concentric lipid bilayers.

Phospholipids are lipids comprising a ‘phosphoric acid’ group. These are lipids consisting of a polar (hydrophilic) “head” and two aliphatic (hydrophobic) “tails”. This family includes in particular phosphatidic acids and phosphoglycerides. The physico-chemical properties of phospholipids depend both on the nature of the polar molecule of the hydrophilic head, and the nature of the aliphatic chains (fatty acids) of their hydrophobic tails.

For the preparation of the liposomal formulation according to the invention, different types of phospholipids can be used.

For example, liposomal formulations shown in patent application WO 2013/129936 are suitable for implementing the present invention.

Without limitation, the following phospholipids could be used in combination:
‘DOPE’ which designates 1,2-Dioleoyl-sn-Glycero-3-Phosphoethanolamine;
‘DSPE’ which designates 1,2-distearoyl-sn-glycero-3-phosphoethanolamine or distearoylphosphatidylethanolamine;
‘PEGXXXX’ which designates polyethylene glycol, with XXXX indicating the molecular weight thereof; in particular PEG350 and PEG5000 can in particular be used;
‘DSPE-PEGXXXX’ which designates 1,2-distearoyl-sn-glycero-3-phosphoethanolamine-N-[methoxy-(polyethylene glycol)-XXXX].
cholesterol (3ß-Hydroxy-5-cholestene, 5-Cholesten-3ß-ol).

According to a particular implementation of the invention, the liposomal formulation consists of phospholipids with the composition: DOPE:DSPE-PEG 5000:DSPE-PEG-350:Cholesterol (54:8:8:30% mol).

According to another particular implementation of the invention, the liposomal formulation consists of phospholipids with the composition:
18:1 (delta9-Cis) DSPE (DOPE)
18:0 PEG5000 DSPE (ammonium salts)
18:0 PEG350 DSPE (ammonium salts), and
Cholesterol.

### Bacterial Lipopolysaccharide (LPS)

The liposomal formulation according to the invention exclusively contains a bacterial lipopolysaccharide, namely contains no other active compound, and in particular contains no antigen, except for the antigen O being a component of LPS.

Within the scope of the present invention, LPS is used as an agent stimulating the innate immune system, and not as an adjuvant enabling the specific immune response to a particular antigen to be increased.

LPS, a component of the external membrane of Gram-negative bacteria, comprises three molecular entities bound to each other by covalent bonds:
lipid A;
the “nucleus” consisting of an oligosaccharide;
and the antigen O.

The lipid A which is the most immunostimulatory part of LPS is highly conserved from one bacterial species to the other, the nucleus is of a very low variability, and the antigen O is specific to the bacterial species from which comes the LPS.

Without limitation, Gram-negative bacteria comprise in particular the following families:
Enterobacteriaceae family:
   Salmonella genus
   Escherichia genus, for example Escherichia coli
   Yersinia genus
Vibrionaceae family:
   Vibrio genus example: Vibriocholerae (responsible for cholera)
Pseudomonadaceae family:
   Pseudomonas genus
Neisseriaceae family
   Neisseria genus example: Neisseria meningitidis (responsible for the bacterial meningitis)
Rhizobiaceae family
   Agrobacterium genus example: Agrobacterium tumefaciens
   Rhizobium genus example: Rhizobium rhizogenes
Alcaligenaceae family
   Bordetella genus

According to a particular implementation of the invention, the LPS contained in the liposomal formulation comes from a bacterium of the Enterobacteriaceae family, excluding the Salmonella enterica species.

According to another particular implementation of the invention, the LPS contained in the liposomal formulation comes from a bacterium of the Enterobacteriaceae family, in particular of the Escherichia genus, and in particular of Escherichia coli species.

The phrase “LPS from a bacterium” indicates that said LPS molecule has the physico-chemical characteristics of the LPS naturally occurring in the external membrane of said bacterium. The characteristics are in particular the nature of fatty acids constituting the lipid A.

The LPS can be a naturally occurring LPS or a synthetic LPS.

More particularly, a naturally occurring LPS can be purified from a bacterial membrane, by techniques well known to those skilled in the art, or even can be obtained in a purified form from a company such as SIGMA-ALDRICH.

A synthetic LPS could be obtained by any synthesis technique known to those skilled in the art.

The LPS used can be encapsulated in liposomes in its entire form (lipid A+nucleus+antigen O) or in an incomplete form, only a LPS fraction being used.

According to a particular aspect of the invention, the LPS encapsulated is used as an incomplete form, that is in the form of a LPS fraction. More particularly, the LPS exclusively consists of its antigen O, or its lipid A, or even a combination of both.

The lipid A of the LPS from the bacterial species Escherichia coli is a ß-1-6 glucosamine dimer.

More particularly, the LPS encapsulated can consist of its lipid A used in the form of monophosphoryl lipid A.

It is intended that LPS can be modified, in particular in order to inhibit or potentiate its immunostimulatory properties. The LPS could also be a LPS that underwent a modification for the purposes of modifying its solubility or toxicity properties.

According to a particular aspect of the invention, in the liposomal formulation for use in therapy, in particular in anti-tumour therapy, the LPS encapsulated in the liposomes is a modified LPS.

### Preparation and Characterisation of the LPS Liposomal Formulation

Any technique known to those skilled in the art can be used to prepare a liposomal formulation exclusively containing a LPS.

It is intended that the phrase “exclusively a LPS” indicates that the liposomal formulation only comprises LPS molecules, excluding any other active compound and/or antigen.

The LPS encapsulated comes from some bacterial species, for example comes from Escherichia coli.

According to a particular implementation of the invention, said formulation exclusively comprises LPS molecules, wherein these molecules can be of different bacterial origins: thus, this can be one, two, three or even more LPS of different bacterial origins. For example, the liposomal formulation can comprise a LPS from E. coli, a LPS from a bacterium of the Salmonella genus and a LPS from a bacterium of the Pseudomonas genus, said LPS molecules being in a complete or incomplete form.

In other words, the pharmaceutical formulation consists of at least one bacterial LPS encapsulated in liposomes.

As is shown in the section of examples, the phrase “liposomal formulation exclusively containing a LPS” indicates that the LPS is property incorporated/encapsulated in the liposomal particles, and not that there is just a simple juxtaposition in a medium here.

According to a particular aspect, the method for preparing the liposomal formulation comprises successive freeze/thaw steps.

According to another aspect of the invention, the method for preparing the liposomal formulation comprises a step of sterilising the liposomal formulation.

According to yet another aspect of the invention, the method for preparing the liposomal formulation comprises a step of filtering liposomes, in order to obtain liposomal particles with a homogeneous size.

According to a preferred aspect of the invention, said liposomal formulation consists of liposomal particles with a homogeneous size.

According to a preferred implementation of the invention, the liposomal formulation is suitable for systemic administration.

### Pharmaceutical Composition

The present invention also relates to a pharmaceutical composition comprising, in an acceptable pharmaceutical medium, at least one liposomal formulation exclusively containing a lipopolysaccharide.

In other words, said pharmaceutical composition comprises, in a pharmaceutically acceptable medium, at least one liposomal formulation consisting of a bacterial lipopolysaccharide encapsulated in liposomes.

For the purposes of the invention, a pharmaceutically acceptable medium designates a vehicle enabling the liposomal formulation to be preserved and administrated, and optionally excipients, the administration of which to an individual or animal is not accompanied with significant deleterious effects, and which are known to those skilled in the art.

A pharmaceutical composition according to the invention can comprise any pharmaceutical excipient necessary, such as buffer agents or agents to adjust pH or isotonicity, or even wetting agents. A pharmaceutical composition according to the invention can also comprise one or more anti-oxidant agents, and/or one or more preservatives.

A liposomal formulation, or a pharmaceutical composition as described above, can be administrated by any suitable way, such as the oral, buccal, sublingual, ophthalmic, rectal, topical way, or by parenteral way, in particular by an intraperitoneal, intradermal, subcutaneous, intravenous or intramuscular way.

According to a preferred implementation of the invention, the liposomal formulation or the pharmaceutical composition as described above is adapted to a systemic administration.

A pharmaceutical composition according to the invention can be formulated for an oral administration, as a tablet, a capsule or a hard gelatine capsule, with a sustained or controlled release, a pill, a powder, a solution, a suspension, a syrup or an emulsion.

According to another embodiment, a pharmaceutical composition according to the invention can be prepared for a parenteral administration, as an injectable.

A pharmaceutical composition according to the invention can be sterilised by any known conventional method, such as filtration. The resulting aqueous solution can be conditioned to be used as such, or be lyophilised. A lyophilised preparation can be combined with a sterile solution before use.

According to a preferred implementation of the invention, the pharmaceutical composition comprises an efficient amount of a liposomal formulation exclusively containing a LPS.

An efficient amount of such a formulation corresponds to an amount which induces the desired response, that is a therapeutic effect, and more specifically an anti-proliferative effect to tumour cells. The efficient amount can depend on a parameter or a plurality of parameters, such as the administration way, the single dose or multiple dose administration, the patient's characteristics, which encompasses age, physical condition, height, weight and the presence of conditions in addition to that treated. These parameters and their influences are well known to those skilled in the art and can be determined by any known method.

The present invention also relates to said pharmaceutical composition, for use as a drug, and more particularly for use as an anti-tumour therapy.

### Administration

The liposomal formulation exclusively containing a LPS, or a pharmaceutical composition comprising it, for use as an anti-tumour therapy, could be administrated to a patient with cancer according to any techniques known to those skilled in the art.

In particular, said liposomal formulation or pharmaceutical composition comprising it could be administrated in one dose, or multidose on a continuous period.

According to a particular aspect of the invention, the liposomal formulation for use as described above, or a pharmaceutical composition comprising it, is administrated once weekly to a patient with cancer.

According to a preferred implementation of the invention, said liposomal formulation for use as described above is administrated by systemic pathway, that is the formulation will travel the blood pathway of the patient to reach its target cells. More precisely, said liposomal formulation could be administrated by the digestive pathway or parenteral pathway.

The present invention also relates to a method for therapeutic treating a tumour, comprising administrating to a patient having said tumour, an efficient amount of a liposomal formulation exclusively containing a LPS.

### Pharmaceutical Combination Product

According to a third aspect, the invention relates to a pharmaceutical combination product comprising:
a liposomal formulation exclusively containing a LPS, i.e. consisting of one or more liposomes each encapsulating a bacterial lipopolysaccharide (LPS) as a single active ingredient; and
at least one cytotoxic compound, notably an anti-tumor compound, chosen from the group consisting of: a therapeutic antibody, a chemotherapy agent, and an immunotherapy agent.

For the purposes of the invention, a pharmaceutical combination product designates a set of therapeutic agents used together for the treatment of one pathology, wherein their administration can be simultaneous, separate or sequential. Thus, the therapeutic agents can be either mixed in one therapeutic composition, or be present in one kit but administrated in a completely separate, or sequential way.

The liposomal formulation exclusively containing a LPS will be such as previously defined, and could be in particular:
a formulation containing a LPS from a bacterium of the Enterobacteriaceae family, in particular of the Escherichia genus, and in particular of the Escherichia coli species; and/or
a formulation containing a LPS exclusively consisting of its lipid A.

As previously discussed, a LPS liposomal formulation can be used in therapy either as such, or be combined to another therapeutic agent. This other therapeutic agent is in particular a cytotoxic compound, that is a compound inducing the cell death on cells on which it works.

Advantageously, the cytotoxic compounds are adapted to specifically target some cells, in particular to target the cancer cells, and are therefore designated as “anti-tumor” compounds.

According to one implementation of the invention, said cytotoxic compound is an immunotherapy agent, such as peptides or non-peptide small molecules having an immuno-modulatory and cytotoxic activity; more preferentially, the immunotherapy agent is a therapeutic antibody.

A therapeutic antibody is an antibody capable of specifically recognising cells to be destroyed, in the case of cancer, this is naturally tumour cells. Tumour cells express antigens at their membrane surface, which can be recognised by antibodies directed thereto. The therapeutic antibodies can be coupled to a toxic substance, be capable of inducing lysis and thus death of the tumour cell recognised by the antibody. The therapeutic antibodies can also act by blocking some receptors at the membrane surface of the tumour cells.

These therapeutic antibodies are increasingly used within the scope of anti-tumour therapies. For example, rituximab, a monoclonal antibody specifically binding to the transmembrane antigen CD20, a protein located on the B lymphocytes and expressed in more than 95% of the B cells of the non-Hodgkin lymphoma, can be mentioned. This therapeutic antibody is indicated for treating patients with stage III-IV follicular lymphomas. Trastuzumab, specific to the HER-2 receptor over-expressed by some tumour cells of breast cancer can also be mentioned.

According to a particular implementation of the invention, the therapeutic antibody contained in the combination product is a monoclonal antibody.

According to another particular implementation of the invention, the therapeutic antibody contained in the combination product is a polyclonal antibody.

According to an implementation of the invention, the therapeutic antibody target is chosen from the group consisting of: CD20, CD52, CD3, CD4, CD5, CD8, CD19, CD22, CD38, CD138, HER2, ErbB2, CD1, CD30, CD33, CD52, CD25, vascular endothelial growth factor (VEGF), endothelial growth factor receptor (EGFR), Insulin-like growth factor receptor 1 (IGF1) and CTLA-4.

According to yet another implementation of the invention, the therapeutic antibody is chosen from the group of antibodies consisting of: abciximab, adalimumab, alemtuzumab, atlizumab, basiliximab, belimumab, bevacizumab, brentuximab vedotin, canakinumab, cetuximab, certolizumab pegol, cixutumumab, daclizumab, denosumab, eculizumab, efalizumab, gemtuzumab, golimumab, ibritumomab tiuxetan, infliximab, ipilimumab (MDX-101), muromonab-CD3, natalizumab, necitumumab, obinutuzumab (GA-101), ocaratuzumab (AME-133v), ocrelizumab, ofatumumab, omalizumab, palivizumab, panitumumab, pertuzumab, PR0131921, ranibizumab, rituximab, SBI-087, tocilizumab, TRU-015, tositumomab, trastuzumab, veltuzumab, and any combination of these antibodies.

According to a particular implementation of the invention, the therapeutic antibody is a monoclonal antibody against the B-lymphocyte antigen CD20, a therapeutic target of B-cell malignancies, such as lymphomas and leukemias.

According to a particular implementation of the invention, the therapeutic antibody is Obinutuzumab (GA-101).

It is intended that for the purposes of the invention, the combination product contains at least one therapeutic antibody, and that it can thus comprise a combination of several therapeutic antibodies.

According to another implementation of the invention, the therapeutic antibody is chosen from the group consisting of antibodies used in anti-tumour therapeutic applications, and in particular consisting of the following antibodies: alemtuzumab, belimumab, bevacizumab, brentuximab vedotin, canakinumab, cetuximab, cixutumumab, daclizumab, denosumab, gemtuzumab, golimumab, ibritumomab tiuxetan, ipilimumab (MDX-101), muromonab-CD3, natalizumab, necitumumab, obinutuzumab (GA-101), ocaratuzumab (AME-133v), ocrelizumab, ofatumumab, panitumumab, pertuzumab, PR0131921, ranibizumab, rituximab, SBI-087, tocilizumab, TRU-015, tositumomab, trastuzumab, veltuzumab, and any combination of these antibodies.

According to a preferred implementation of the invention, the therapeutic antibody is an anti-CD20 monoclonal antibody, and preferably is rituximab.

The experimental results shown in FIG. 1 show that:
in untreated mice having a tumour, the tumour volume increases over time;
if the mice are treated by administration of rituximab, the tumour volume still increases over time, but said volume is twice lower, 46 days after the tumour graft, than the tumour volume in control mice;
if the mice are treated with a combination product comprising rituximab and a LPS liposomal formulation, the tumour volume is identical 23 days and 46 days after grafting: the tumour development is thus completely inhibited.

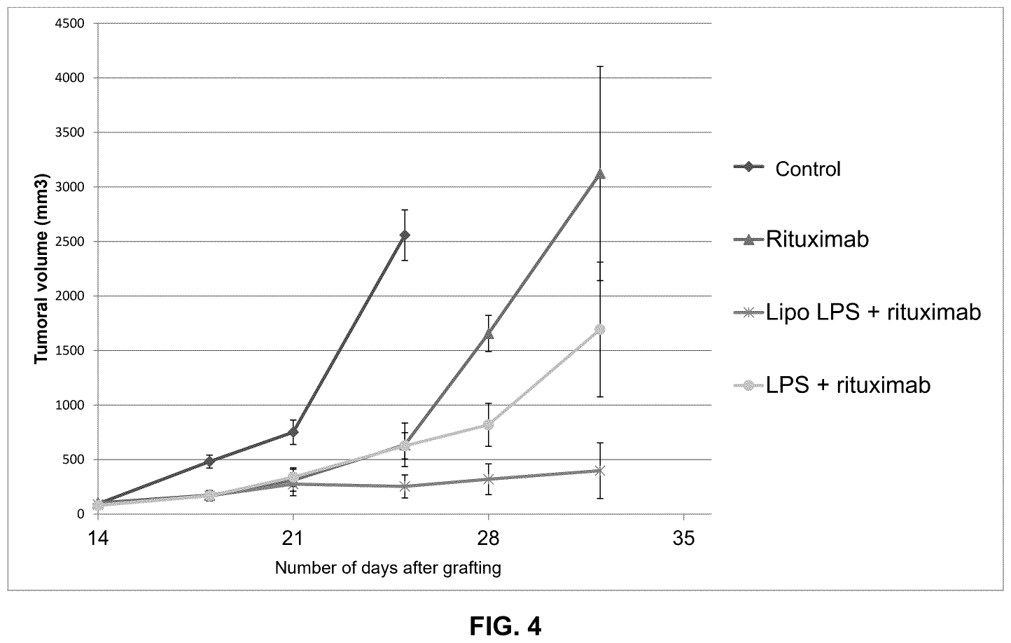

FIG. 4 shows the difference observed between mice treated with this combination product, and mice treated with a combination product comprising rituximab and LPS non-encapsulated in liposomes: the LPS liposomal formulation is more efficient to inhibit tumour development.

According to another implementation of the invention, the cytotoxic compound is chosen from the following compounds:
conventional cytotoxic agents (chemotherapy agents) such as:
   alkylating agents and the like such as cyclophosphamide, busulfan, melphalan, ifosfamide, bendamustine, temozolomide, bleomycin, mechloretamine, carmustine, fotemustine, BCNU;
   platinum derivatives such as cisplatin, oxaliplatin, carboplatin,
   nucleoside analogs, nucleobases and other anti-metabolites such as aracytin, fludarabine, cladribine, clofarabine, gemcitabine, 5-fluorouracile, capecitabine, methotrexate, pemetrexed, 6-mercaptopurine, decitabine, azacytidine, troxacitabine,
   anti-tubulin agents such as vincristine, vinblastine, vindesine, vinorelbine, vinflunnine, paclitaxel, docetaxel, cabazitaxel, epothilones, eribulin, micronised paclitaxel,
   anti-topoisomerase 2 agents such as etoposide, teniposide, irinotecan, camptothecin, doxorubicin, daunorubicin, idarubicin, mitoxantrone, dactinomycin;
targeted therapy agents of the kinase inhibitor type such as imabinib, dasatinib, bosutinib, nilotinib, ponatinib, ibrubinib, idelalisib, afatinib, erlotinib, gefitinib, lapatinib, crizotinib, ruxolitinib, tofacitinib, axitinib, cabozantinib, nindetanib, pazopanib, vandetanib, regorafenib, sorafenib, sunitinib, dabrafenib, trametinib, vemurafenib;
immunomodulators such as thalidomide, lenalidomide, pomalidomide;
apoptosis enablers facilitators such as venetoclax or navitoclax;
anti-tumour agents such as bortezomib, carfilzomib, ixazomib, vorinostat, panobinostat, romidepsin, all-trans retinoic acid, arsenic derivative As2O3, temsirolimus, olaparib, rucaparib, pentostatin, asparaginase, hydroxyurea, and chloraminophene;
immunotherapy agents such as inhibitors of immune checkpoint.

According to a preferred implementation of the invention, the cytotoxic compound is a chemotherapy agent.

It is intended that the abovementioned compounds could be used under their usual form, in particular as salts. For example, etoposide could be used as an epotoside phosphate.

Example 4 of the present application demonstrates that LPS encapsulated in liposomes, used in combination with etoposide (5 μM), enables cell death of cancer cells in vitro to be caused, according to a synergist mode.

Indeed, etoposide, a conventionally used chemotherapy agent, induces cancer cell apoptosis, as expected; on the other hand, the LPS liposomal formulation stimulates macrophages, thus inducing tumour cells lysis; and the combined effect of both components enables a cell death rate much higher than the sum of both effects of each component used separately to be achieved.

In a particular embodiment, the chemotherapy agent is chosen in the group consisting of gemcitabin, irinotecan and bleomycin.

In another embodiment of the invention, the anti-tumor compound is an immunotherapy agent, such as an immune checkpoint inhibitor.

Immune checkpoint are regulators of the immune system. Inhibitors of these checkpoint molecules are targets for cancer immunotherapy. Currently approved immune checkpoint inhibitors block CTLA4, PD-1 and PD-L1.

In a specific embodiment of the invention, the immune checkpoint inhibitor is chosen among the group consisting of inhibitors of PD-1 and inhibitors of PD-L1.

In a more specific embodiment of the invention, the immune checkpoint inhibitor is chosen among the group consisting of antibodies against PD-1 and antibodies against PD-L1.

The present invention also relates to a pharmaceutical combination product as shown above, for use in anti-tumour therapy.

In particular, said pharmaceutical combination product will be used to treat a liquid tumour or a solid tumour.

Said tumour could in particular be chosen from the group consisting of: a breast tumour, a lung tumour, a skin tumour (melanoma), a blood tumour (leukemia), a bone tumour and a lymphoma.

More precisely, the present invention relates to said pharmaceutical combination product for simultaneous, separate or sequential use in anti-tumour therapy. In other words, the use of each component of said combination product could be simultaneous, separate or sequential.

More particularly, it relates to a pharmaceutical combination product as shown above, for simultaneous, separate or sequential use in anti-tumour immunotherapy.

The present invention also relates to a method for therapeutic treating a tumour, comprising administrating to a patient having said tumour, at least one cytotoxic compound and a liposomal formulation exclusively containing a LPS, said administration being made in a simultaneous, separate or sequential way.

More precisely, the present invention relates to a method for treating a tumor comprising administering to a patient having said tumor an effective amount of the pharmaceutical combination product as described above.

The present invention relates also to a kit comprising:
a liposomal formulation exclusively containing a LPS; and
at least one cytotoxic compound such as defined above.

Said cytotoxic compound could be in particular an anti-tumor compound chosen from the group consisting of a chemotherapy agent, a therapeutic antibody and an immunotherapy agent.

### EXAMPLES

### Example 1. Preparation, Analysis and Characterisation of the Liposomal Formulations in which the LPS Molecules (LPS Extracted and Purified from Escherichia coli 055:B5) are Encapsulated: Liposomes-LPS

The formulations of liposomes-LPS have been synthesised according to the following procedure:
lipid composition of the liposomal formulation: DOPE:DSPE-PEG 5000:DSPE-PEG-350:Chol (54:8:8:30% mol)
dissolving the lipids in a chloroform/methanol mixture (9:1)
preparing the lipid film after evaporating the organic solvents. The evaporation is performed using a rotary evaporator
the hydration of the lipid film is performed by adding PBS (buffered saline solution) containing 100 μg LPS/mL in the flask placed in a rotary evaporator, without vacuum.
to improve the encapsulation rate, a freeze (in liquid nitrogen)—thaw (in warm water) series is applied to the suspension of liposomes-LPS.

Extruding the solution of liposomes-LPS enables liposomes with a homogeneous size to be obtained. To this end, the solutions of liposomes-LPS are filtered on 800, 400 and 200 nm polycarbonate membranes. After 5 runs on each filter, the liposome size is homogeneous and the solution of liposomes-LPS is translucent.

The analysis of size (mean diameter), polydispersity (PDI) and charge of liposomal preparations has been performed by dynamic light scattering using a ZetasizerNano-S from Malvern instruments (Worcestershire, UK). The analysis on 3 months of two liposomal preparations allowed stability of these preparations to be demonstrated (table 1).

TABLE 1

| | | | Polydis- | | Zeta |
|---|---|---|---|---|---|
| | Measurements | Size | persity | Osmolarity | potential |
| | made at | (nm) | (PDI) | (mOsm/kg) | (mv) |
| Liposomal | D 0 | 133 | 0.09 | 309 | −3.63 |
| preparation | D 0 + 30 d | 132 | 0.09 | 308 | −3.67 |
| containing | D 0 + 60 d | 133 | 0.111 | 311 | ND |
| LPS | D 0 + 90 d | 134 | 0.098 | 310 | −6.13 |

The in vitro analysis of the interaction of liposomal formulations thus produced (involving a fluorescent component) with human blood white blood cells allowed a strong interaction of liposomes with granulocytes and monocytes to be demonstrated. The interaction with the lymphocytic fraction has not been observed (results not shown).

Additionally, the incubation of the human blood white blood cells with the liposomes-LPS and the analysis by flow cytometry enabled us to emphasise the activation of the phagocyte activity.

The confocal microscopy analysis of the rhodamine B-labelled liposomes-LPS formulations and in which the LPS molecules are coupled to the fluorochrome FITC allowed us to emphasise the incorporation of LPS into liposomal particles.

Finally, the homogeneity of liposomal particles has been validated by electron microscopy after negative staining.

### Example 2. Preclinical Evaluation of the Adjuvant Effect of the LPS Liposomal Formulations on the Rituximab Activity in a RL Cells Xenografted Mice Model

During this experiment, six groups of Scid CB17 mice have been used. Each group is comprised at six mice.

$$Group 1 = control ( no treatment )$$

$$Group 2 = Empty liposomes$$

$$Group 3 = 0.5 mg / mL LPS$$

$$Group 4 = 30 mg / kg Rituximab$$

$$Group 5 = 0.5 mg / kg Liposomes - LPS$$

$$Group 6 = 0.5 mg / kg Liposomes - LPS + 30 mg / kg Rituximab$$

The monitoring of tumour growth after injecting the different treatments at a rate of one injection per week enabled the rituximab anti-tumour activity potentiator effect to be demonstrated by the preparation of liposomes-LPS. This potentiator effect was reflected in a significant reduction in the tumour growth after the Rituximab and liposomes-LPS combination in comparison with the group of mice treated by the rituximab alone (FIG. 1).

The analysis of the effect of the different treatments on the spleen size after sacrificing the animals enabled a significant increase in the spleen size and weight to be emphasised in the case of animals treated by the liposomes-LPS and the Rituximab and Liposomes-LPS combination in comparison with animals from other groups (FIG. 2).

NK cells are innate immunity cells which are known to play an important role in anti-tumour responses.

The flow cytometry analysis of the effect of the different treatments on the white blood cell sub-populations at the spleen enabled the significant increase in the number of Natural Killer (NK) cells to be emphasised in the spleen of animals treated by the liposomes-LPS and Rituximab and Liposomes-LPS combination in comparison with animals from other groups (FIG. 3 shows the percentage of NK cells relative to all the white blood cells in the sample).

FIG. 4 shows, in the same animal model of SCID mice grafted with RL lymphoma cells, the compared effects obtained with the administration, once a week, of:
rituximab (30 mg/kg)
rituximab and “conventional” LPS of E. coli (0.5 mg/kg)
rituximab and LPS liposomal formulation, at the same doses as previously.

The latter rituximab and encapsulated LPS combination enables the best inhibition in the development of the tumour after its grafting to be achieved.

### Example 3. Anti-Tumour Action of the LPS Liposomal Formulation in Xenograft Models of Solid Tumours

The anti-tumour action of the LPS liposomal formulation has also been shown in another animal model of tumours, SCID mice xenografted with breast cancer tumour cells: the MDA-MB-231 cells.

The results shown in FIG. 5 show the progression of the tumour volume over time, as a function of the treatments administrated to the mice:
Group 1: untreated control mice (3 mice);
Group 2: mice treated by the intravenous administration of the LPS liposomal formulation.

The tumour growth monitoring after injecting the different treatments at a rate of one injection per week enabled the anti-tumour activity of the liposomes-LPS preparation to be demonstrated. This activity was reflected by a significant reduction in the tumour growth after injecting the liposomes-LPS in comparison with the group of untreated mice.

### Example 4. In Vitro Effects of a Combination Product According to the Invention on the Cell Death Rate of SaOS Cells

The SaOS cells are rat osteosarcoma cells. This is a malignant bone tumour. These cells can be cultured according to a 3D spheroid model adapted to tumour cells.

THP-1 are human monocytic cells derived from an acute monocytic leukemia.

To perform cellular death monitoring of these SaOS cells, the fluorescent product IncuCyte® Caspase-3/7 has been used to evaluate the number of apoptosis cells.

The cells have been treated according to the protocol shown in the following table 2:

TABLE 2
Treatment protocol of the THP-1 effector
cells and the target SaOS cells

| Days | THP-1 effector cells | Target Saos cells |
|---|---|---|
| D 0 | Activation of THP-1 | The Saos cells are placed in |
| | cells in | 96-well |
| | macrophages: | plates (2500 cells/well) to |
| | treatment with 150 nM | initiate spheroid growth |
| | PMA (phorbol | |
| | myristate acetate) | |
| | for 24 hours | |
| D 1 | Change of culture | |
| | medium | |
| D 2 | Activation of THP-1 | |
| | with an incubation | |
| | for 16 hours in the | |
| | presence of 100 ng/ | |
| | Lipo-LPS | |
| D 3 | | Mix of the activated THP-1 |
| | | cells and target cells: |
| | | target/effector |
| | | ratio = ⅓ |
| | | +5 μM etoposide |
| | | +2.5 mM final Green |
| | | casp 3/7 fluorescent |
| | | compound |
| | | Incubation 30 minutes at |
| | | room temperature and then |
| | | observation under microscope |
| | | after development by |
| | | Incucyte ®, every three hours |

The results are shown in FIG. 6, in fluorescence arbitrary units.

The control cells have a small residual apoptosis rate.

Cells treated with epotoside have an apoptosis rate significantly increased with respect to the control (p<0.05). Cells treated with a LPS liposomal formulation have a similar rate.

The association of both compounds: epotoside and liposomal formulation, enables an apoptosis rate significantly increased with respect to that obtained for each of the compounds used alone to be achieved, thus demonstrating the synergist effect of both compounds on the target cancer cells.

### Example 5. Preclinical Evaluation of the Anti-Tumor Activity of the LPS Liposomal Formulation (HEPHA-440) and its Adjuvant Effect on the Activity of Monoclonal Antibodies Against Mouse CD20 (Anti-CD20 mAb—Clone 18B12) in a Syngenic Mouse Model of B-Cell Lymphoma (A20)

During this experiment, four groups of BALB/c mice have been used. Each group is comprised of eight mice.

$$Group 1 = control ( Empty liposomes )$$

$$Group 2 = 1.25 mg / kg ( i . v . ) HEPHA - 440$$

$$Group 3 = 12.5 mg / kg ( i . p . ) anti - CD 20 mAbs ( 18 B 12 )$$

$$Group 4 = 1.25 mg / kg ( i . v . ) HEPHA - 440 + 12.5 mg / kg ( i . p . ) anti - CD 20 mAbs ( 18 B 12 )$$

The monitoring of tumour growth after administration the different treatments at a rate of one injection per week for three weeks enabled to demonstrate:
the antitumor activity of the liposome-LPS preparation (HEPHA-440),
the potentiation of the antitumor activity of the anti-CD20 mAb by HEPHA-440.

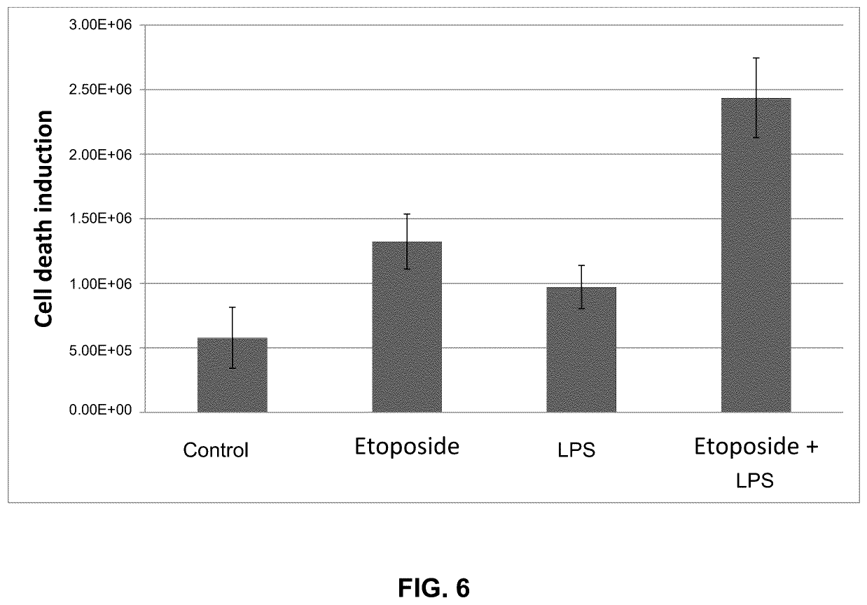

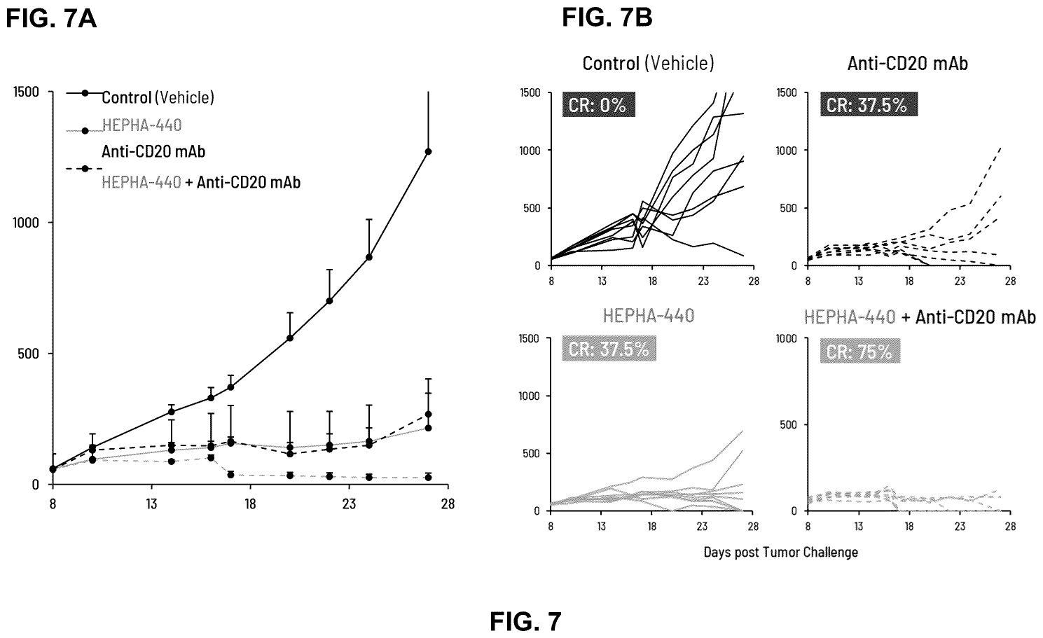

The antitumor activity of HEPHA-440 was reflected by a significant reduction (p<0.001) of the tumour growth (FIG. 7A) and an increase of the percentage of mice in complete remission (37.5% vs. 0%—FIG. 7B) in the group of mice treated with HEPHA-440 in comparison with the control group.

The potentiator effect of HEPHA-440 was reflected in a reduction (p<0.09) of the tumour growth after the administration of the anti-CD20 mAbs and HEPHA-440 combination in comparison with the group of mice treated by the anti-CD20 mAbs alone (FIG. 7A). This was also reflected in an increase of the percentage of mice in complete remission after the combined administration of the anti-CD20 mAbs and HEPHA-440 in comparison with the group of mice treated by the anti-CD20 mAbs alone (37.5% vs. 75% respectively—FIG. 7B).

### Example 6. Preclinical Evaluation of the Adjuvant Effect of the LPS Liposomal Formulation (HEPHA-440) on the GA101 Activity in a RL Cells Xenografted Mice Model

During this experiment, three groups of Scid CB17 mice have been used. Each group is comprised of six mice.

$$Group 1 = control ( No treatment )$$

$$Group 2 = 0.5 mg / kg ( i . v . ) HEPHA - 440$$

$$Group 3 = 30 mg / kg ( i . p . ) GA 101$$

$$Group 4 = 0.5 mg / kg ( i . v . ) HEPHA - 440 + 30 mg / kg ( i . p . ) GA 101$$

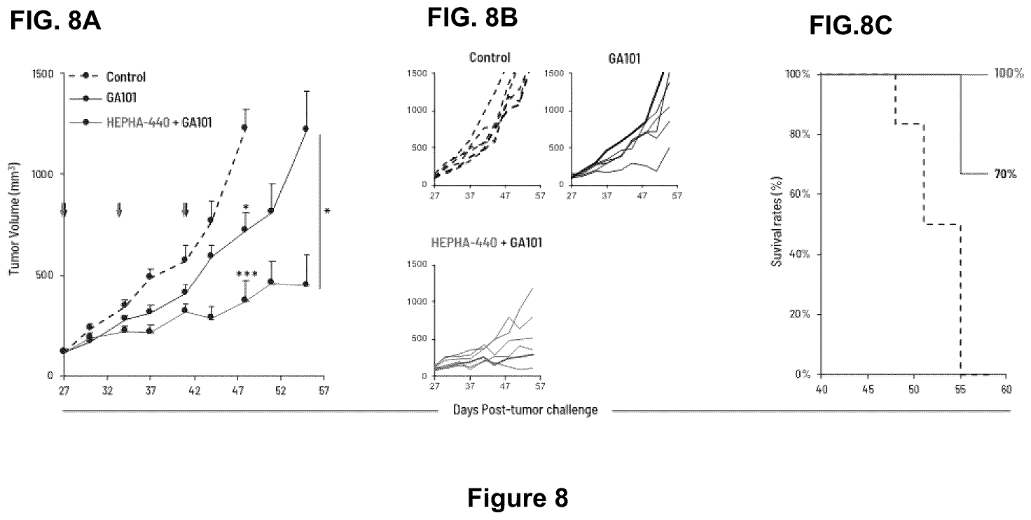

The monitoring of tumour growth after injecting the different treatments at a rate of one injection per week for three weeks enabled to demonstrate the potentiation of the GA101 antitumor activity by the liposome-LPS preparation (HEPHA-440). This potentiator effect was reflected by a significant reduction (p<0.05) of the tumour growth after the combined administration of the GA101 and HEPHA-440 in comparison with the group of mice treated by the GA101 alone (FIGS. 8A & B). This potentiator effect was also reflected by an increase of the survival rate at day 58 in the group of mice treated with the combination of GA101 and HEPHA-440 compared to the group of mice treated with GA101 alone (100% vs. 70% respectively—FIG. 8C).

### Example 7. Preclinical Evaluation of the Anti-Tumor Activity of HEPHA-440 and its Adjuvant Effect on the Activity of a Monoclonal Antibody Against Mouse PD1 (Anti-PD1 mAb—Clone RPM1-14) in a Syngenic Mouse Model of Colorectal Carcinoma (MC38)

During this experiment, four groups of C57BL/6 mice have been used. Each group is comprised of six mice.

$$Group 1 = control ( No treatment )$$

$$Group 2 = 12.5 mg / kg ( i . p . ) anti - PD 1 mAb$$

$$Group 3 = 0.5 mg / kg ( i . v . ) HEPHA - 440$$

$$Group 4 = 0.5 mg / kg ( i . v . ) HEPHA - 440 + 12.5 mg / kg ( i . p . ) anti - PD 1 mAb$$

The monitoring of tumour growth after administration the different treatments at a rate of one injection per week for three weeks enabled to demonstrate:
the antitumor activity of the liposome-LPS preparation (HEPHA-440).
the potentiation of the antitumor activity of the anti-PD1 mAb by HEPHA-440.

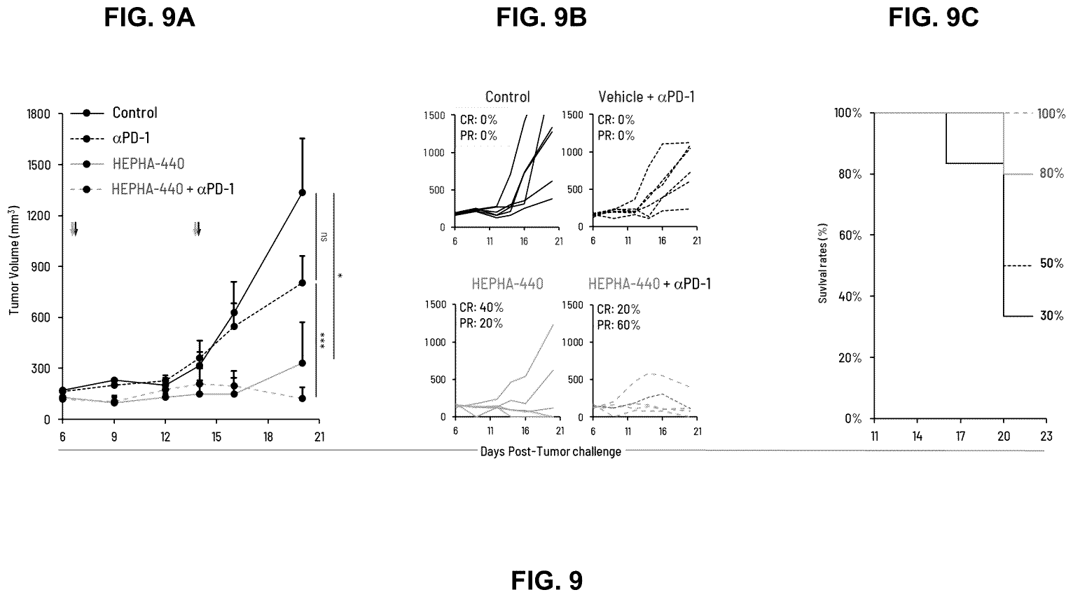

The antitumor activity of HEPHA-440 was reflected by a significant reduction (p<0.05) of the tumour growth (FIG. 9A) and an increase of the percentage of mice in complete or partial remission (CR:40%/PR:20% vs. CR:0%/PR:0%—FIG. 9B) as well as a an increase of the survival rate (80% vs. 30% at day 22—FIG. 9C) in the group of mice treated with HEPHA-440 in comparison with the control group.

The potentiator effect of HEPHA-440 was reflected by a significant reduction of the tumour growth (p<0.001) after the administration of the anti-PD1 mAb and HEPHA-440 combination in comparison with the group of mice treated by the anti-PD1 mAb alone (FIG. 9A). The potentiator effect was also reflected by an increase of the percentage of mice in complete or partial remission after the combined administration of the anti-PD1 mAb and HEPHA-440 in comparison with the group of mice treated by the anti-PD1 mAb alone (CR:20%/PR:60% vs. CR:0%/PR:0% respectively—FIG. 9B). The potentiator effect was also reflected by an increase of the survival rate after the combined administration of the anti-PD1 mAb and HEPHA-440 in comparison with the group of mice treated by the anti-PD1 mAb alone (100% vs. 50% at day 22—FIG. 9C).

### Example 8. In Vitro Evaluation of the Impact of HEPHA-440 on the Anti-Tumor Activity of Chemotherapy Agents on Human Osteosarcoma Cells (Saos-2) and Mouse Colon Adenocarcinoma Cells (MC38)

The SaOS-2 cells are human osteosarcoma cells. This is a malignant bone tumour. These cells can be cultured according to a 3D spheroid model adapted to tumour cells.

THP-1 are human monocytic cells derived from an acute monocytic leukemia.

The cells have been treated according to the protocol shown in the following table 3:

TABLE 3
Treatment protocol of the THP-1 effector cells and the target SaOS2 cells

| Days | THP-1 effector cells | Target Saos-2 cells |
|---|---|---|
| D0 | Activation of THP-1 cells in | The Saos-2 cells were placed in 96- |
| | macrophages: treatment | well plates (2500 cells/well) to |
| | with 150 nM PMA (phorbol | initiate spheroid growth |
| | myristate acetate) for | |
| | 24 hours | |
| D1 | Change of culture medium | |
| D2 | Stimulation of THP-1 with an | |
| | incubation for 16 hours in | |
| | the presence of 250 ng/ml of | |
| | Lipo-LPS (HEPHA-440) or | |
| | medium (non activated) | |
| D3 | | Mix of the stimulated |
| | | (+HEPHA-440) |
| | | or non stimulated THP-1 |
| | | cells and target cells: |
| | | target/effector |
| | | ratio = ⅓ |
| | | +/−200 nM doxorubicin |
| | | After 24 h of incubation, |
| | | size of Saos-2 |
| | | spheroids was evaluated using |
| | | IncuCyte S3 spheroid analysis |
| | | software module. |

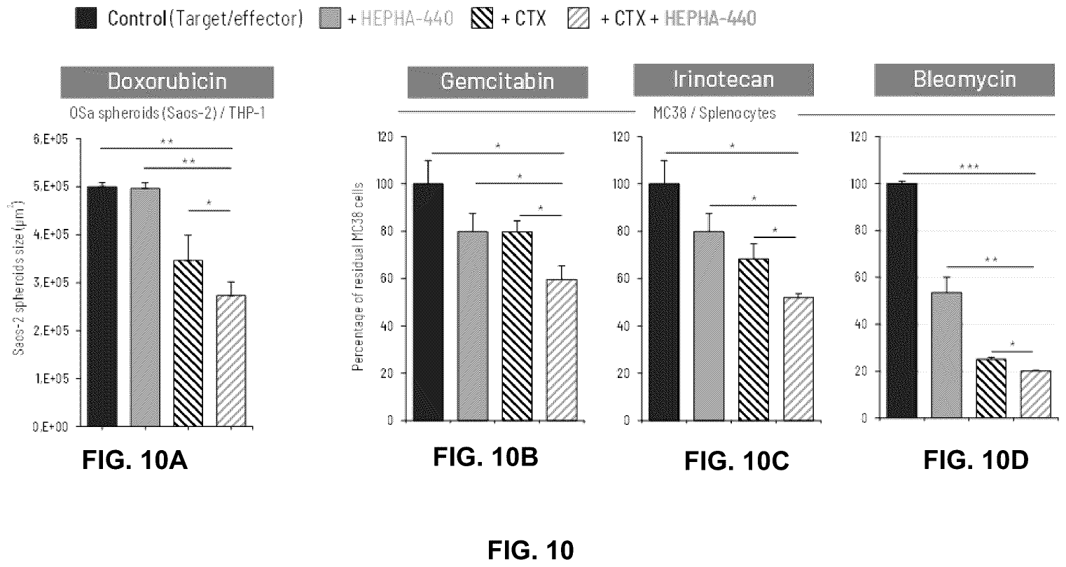

Mean size (μm²) of Saos-2 spheroids in each the conditions are presented in FIG. 10A.

Doxorubicin significantly decreased (p<0.01) the size of Saos-2 spheroids with respect to the control unlike HEPHA-440 stimulated THP-1 effector cells alone.

The association of both compounds (doxorubicin and HEPHA-440) significantly decreased (P<0.05 vs. Doxorubicin alone and P<0.01 vs. other groups) the size of Saos-2 spheroids with respect to that obtained for each of the compounds used alone to be achieved, thus demonstrating the synergist effect of both compounds on the target cancer cells.

MC38 cells in 2D cultures in 96 well plates were incubated with fresh murine splenocytes (Ratio target (MC38)/Effector (Splenocytes): 1/10) in the presence or absence of HEPHA-440 (10 μg/ml) and/or chemotherapeutic agents (gemcitabin, irinotecan, or bleomycin). MC38 cells only incubated with splenocytes were used as control. After 72 of incubation, an MTT cell viability assay was performed to evaluate residual viable MC38 cells, after elimination of splenocytes by washing.

The percentage of viable MC38 cells in each condition are presented in FIG. 10B (Gemcitabin), FIG. 10C (Irinotecan), and FIG. 10C (Bleomycin).

MC38 viability was significantly decreased in presence of HEPHA-440 alone (P<0.05), as well as in presence of Gemcitabin (P<0.05), Irinotecan (P<0.05), and Bleomycin (P<0.001) alone with respect to the control.

The association of HEPHA-440 with each of the three individual chemotherapeutic agents (Gemcitabin, Irinotecan, or Bleomycin) significantly reduced (P<0.05) the viability of MC38 cells compared to that obtained for each of the compounds used alone, demonstrating the synergistic effect of HEPHA-440 with each chemotherapeutic agents on the target cancer cells.

### REFERENCES

### Patent

WO 2013/129936

### Bibliographic Citations

Bakouche O, Koff W C, Brown D C, Lachman L B. Interleukin 1 release by human monocytes treated with liposome-encapsulated lipopolysaccharide. J. Immunol. 1987 Aug. 15; 139(4):1120-6.
Dijkstra J, Mellors J W, Ryan J L. Altered in vivo activity of liposome-incorporated lipopolysaccharide and lipid A. Infect Immun. 1989 November; 57(11):3357-63.
Neidhart J, Allen K O, Barlow D L, Carpenter M, Shaw D R, Triozzi P L, Conry R M. Immunization of colorectal cancer patients with recombinant baculovirus-derived KSA (Ep-CAM) formulated with monophosphoryl lipid A in liposomal emulsion, with and without granulocyte-macrophage colony-stimulating factor. Vaccine. 2004 Jan. 26; 22(5-6):773-80.

1. A pharmaceutical combination product comprising:
a liposomal formulation consisting of one or more liposomes each encapsulating a bacterial lipopolysaccharide (LPS) as a single active ingredient; and
at least one anti-tumor compound chosen from the group consisting of: a therapeutic antibody, a chemotherapy agent, and an immunotherapy agent.

2. The pharmaceutical combination product according to claim 1, characterised in that the anti-tumor compound is a therapeutic antibody.

3. The pharmaceutical combination product according to claim 2, wherein the therapeutic antibody is a monoclonal antibody against CD20.

4. The pharmaceutical combination product according to claim 3, wherein the therapeutic antibody is obinutuzumab (GA-101).

5. The pharmaceutical combination product according to claim 1, wherein the anti-tumor compound is a chemotherapy agent.

6. The pharmaceutical combination product according to claim 5, wherein the chemotherapy agent is chosen in the group consisting of gemcitabin, irinotecan, and bleomycin.

7. The pharmaceutical combination product according to claim 1, wherein the anti-tumor compound is an immunotherapy agent.

8. The pharmaceutical combination product according to claim 7, wherein the immunotherapy agent is an immune checkpoint inhibitor.

9. The pharmaceutical combination product according to claim 8, wherein the immune checkpoint inhibitor is chosen from the group consisting of antibodies against PD-1 and antibodies against PD-L1.

10. A method for treating a tumor comprising administering to a patient having said tumor an effective amount of the pharmaceutical combination product of claim 1.

11. The method according to claim 10, wherein said tumor is selected from the group consisting of liquid tumours and solid tumours.

12. The method of claim 10, wherein said tumor is chosen from the group consisting of: a breast tumour, a lung tumour, a melanoma, a leukaemia, a bone tumour, and a lymphoma.

13. The method of claim 10, wherein the administration of said liposomal formulation and said anti-tumor compound is simultaneous, separate, or sequential.

14. The method of claim 10, wherein the liposomal formulation is suitable for systemic administration.
